(19) United States
(12) Patent Application Publication
(10) Pub. No.: US 2024/0277345 A1
(43) Pub. Date: Aug. 22, 2024

(54) POINT-PRESSURE APPLICATION SYSTEM

(71) Applicant: 6:8 Medical Solutions LLC, Myrtle Beach, SC (US)

(72) Inventors: Uriah Shane Popp, Havre De Grace, MD (US); Paul Clifton Stultz, Myrtle Beach, SC (US)

(73) Assignee: 6:8 Medical Solutions LLC, Myrtle Beach, SC (US)

(21) Appl. No.: 18/628,292

(22) Filed: Apr. 5, 2024

Related U.S. Application Data
(63) Continuation-in-part of application No. 17/336,135, filed on Jun. 1, 2021.
Application No. 17/336,135 is a continuation of application No. 16/364,105, filed on Mar. 25, 2019, now Pat. No. 11,020,127.
(60) Provisional application No. 63/466,667, filed on May 15, 2023.
(60) Provisional application No. 62/648,311, filed on Mar. 26, 2018.

(51) Int. Cl. A61B 17/132; A61B 17/12

(52) U.S. Cl. CPC A61B 17/1325; A61B 17/12; A61B 2017/12004; A61B 17/132

(57) ABSTRACT

A targeted pressure device for providing point or proximal pressure to a targeted site in order to help occlude open blood vessels includes a base plate and two or more anchoring members. The base plate has a first surface configured to face towards the targeted site and a second surface opposite of the first surface. The anchoring members are fixedly coupled to the second surface of the base plate and extend perpendicular to the base plate. Each one of the anchoring members has a narrow distal portion and a wide proximal portion having a proximal end that is a fixed distance away from the base plate. The anchoring members are configured for engaging with a wrapping member that is used to attach the targeted pressure device to the targeted site.

### CROSS-REFERENCE TO RELATED APPLICATIONS

This application claims priority to U.S. patent application Ser. No. 17/336,135, filed Jun. 1, 2021, entitled “Point-Pressure Application System,” which claims priority to U.S. Pat. No. 11,020,127 filed on Mar. 25, 2019, which claims priority to U.S. Provisional Patent Application Ser. No. 62/648,311 filed Mar. 26, 2018. This application further claims priority to U.S. Provisional Patent Application Ser. No. 63/466,667 filed May 15, 2023. The entire contents of the above-listed applications are incorporated herein by reference.

### BACKGROUND

### Field of the Art

The present invention pertains to a system for applying point pressure on a wound site. More specifically, the present disclosure pertains to a point pressure applicator that may be used for limiting hemorrhage by occluding open wounds, blood vessels, etc., or for other medical and/therapeutic uses.

### Discussion of the State of the Art

Hemorrhages that are caused by open blood vessels or wounds represent a significant danger to patients because they can result in exsanguination. Short term arterial blood loss can be irreversible and may lead to death if left unaddressed. The traditional method for treating hemorrhages is to apply a tourniquet, which wraps around a patient's injured limb and applies constrictive force around the patient's entire limb in an effort to occlude damaged blood vessels and prevent further bleeding.

However, traditional tourniquet systems can not be applied to truncal (or non-limbic) portions of a patient's body. For example, traditional tourniquets are ineffective at stopping bleeding in the pelvic region, the chest, the shoulder, the stomach, etc. Moreover, traditional tourniquets, when applied properly often damage surrounding tissues because of the excessive pressure that may be required in order to achieve vessel occlusion. This ancillary damage may be exacerbated if nearby organs have already suffered trauma. As such, traditional tourniquets sometimes are avoided and some physicians regard them as doing more harm than good.

Some prior art systems have attempted to alleviate this problem by applying point pressure directly to the injured vessels. These systems apply downward pressure directly on the injured vessel instead of applying constricting force around the entire limb. These systems are much more effective at stopping hemorrhages to junctional locations, and tend to cause less damage to nearby areas. However, these point pressure systems also suffer from significant other limitations. For one, these systems cannot be deployed very quickly. Generally, users or medical care providers must receive extensive training to use these devices, and they are not intuitive in design. They must be properly positioned with a series of screws, air bladders, and straps making repositioning very timely. Thereafter, medical care providers must apply pressure by screwing in a point-pressure applicator, or pump an air bladder, or using a pneumatic pressure application system, which can be affected during aeromedical evacuation from atmospheric pressure. The entire process can take valuable time, or have limitations, which may be critical in saving the patient's life.

However, presently available point-pressure systems are unusable in a variety of different field or transport situations. For example, presently available point-pressure systems rely on long, vertical rods with a counter foot plate that apply downward pressure to one or more blood vessels. However, these vertical rods with foot plate, because they rely on a stable laying surface, may fall out of place if, for example, a patient moves, if the patient is moved from one location to another, if the rods is accidentally knocked over by a nearby person, or if the patient is being transported in a vehicle that is moving, such as a transport truck or an aerial vehicle. Pneumatic pressure systems and air bladders are similarly limited. For example, air bladders are affected by changes in atmospheric pressure (such as during aeromedical evacuation environments) and the materials used in these devices do not fare well in extreme temperature storage variances, such as the ones that military equipment must endure.

These problems are exacerbated by the fact that it is nearly impossible to use traditional point-pressure systems in a manner that permits the pressure rod to extend vertically above the wound. Human subjects, because of their natural biology, have asymmetric shapes and rounded contours. As a result, in practice, the pressure rod in point-pressure systems rarely extends vertically above a wound. Instead, the pressure rods typically extend above the wound at an angle. This makes the entire system more unstable and more prone to getting knocked over in busy environments.

Finally, traditional tourniquet systems and the newer point-pressure systems are not designed for self-application. These systems require medical professionals to use two hands simultaneously to apply the devices over wounds and to apply pressure once the device is in place. However, single-handed use—and the ability to apply pressure to the patient's own injury—is a critical requirement for military use devices because medical help may not be readily available.

### SUMMARY

The present invention is a point-pressure application system that overcomes the limitations described above. More specifically, the present invention is for a targeted pressure device for providing point or proximal pressure to a targeted site. The targeted pressure device includes a base plate having a first surface configured to face towards the targeted site and a second surface opposite of the first surface, wherein the second surface is configured to face away from the targeted site. The first surface of the base plate may have a rough texture for providing grip to the targeted site. The base plate may be curved such that the first surface is convex and the second surface is concave.

The targeted pressure device further includes at least two anchoring members fixedly coupled to the second surface of the base plate and extending in a substantially perpendicular direction relative to the base plate. The anchoring members are configured for engaging with a wrapping member that is used to attach the targeted pressure device to the targeted site. The base plate and the at least two anchoring members may be formed as a single, unitary piece.

Each one of the anchoring members includes a narrow distal portion having a distal end fixedly coupled to the second surface of the base plate, and a wide proximal portion having a proximal end that is a fixed distance away from the base plate. The wide proximal portion may include a bracket that is C-shaped or U-shaped and configured for holding the wrapping member in place. In another example, the wide proximal portion may include a horizontal bar that is configured for holding the wrapping member in place. The wide proximal portion may include protruding teeth for gripping the wrapping member. The narrow distal portion may include a smooth surface that allows the wrapping member to move relative thereto.

In one example, the targeted pressure device may include four anchoring members, wherein a first anchoring member, a second anchoring member, a third anchoring member, and a fourth anchoring member are coupled to a first side of the base plate, a second side of the base plate, a third side of the base plate, and a fourth side of the base plate, respectively. The first anchoring member and the second anchoring member may be positioned opposite of each other, and the third anchoring member and the fourth anchoring member may be positioned opposite of each other. The proximal ends of the first anchoring member and the second anchoring member may be a first fixed distance away from the base plate, and the proximal ends of the third anchoring member and the fourth anchoring member may be a second fixed distance away from the base plate. The second distance may be different from the first distance.

The targeted pressure device may further include a pressure-enhancing attachment configured to attach to the first surface of the base plate, such that a distance between the pressure-enhancing attachment and the proximal ends of the anchoring members may be greater than the fixed distance between the base plate and the proximal ends of the anchoring members. The pressure-enhancing attachment and the base plate may be configured to attach together with a friction fit attachment, snap fit attachment, adhesive, suction, tongue and groove, hook and loop fasteners, magnets, clamps, or clips. For example, the base plate may include an aperture having an aperture size and aperture shape, and the pressure-enhancing attachment may include a protrusion having a protrusion size and a protrusion shape that are substantially the same as the aperture size and the aperture shape, respectively, so that the pressure-enhancing attachment is coupled to the base plate with a friction fit between the aperture and the protrusion. The pressure-enhancing attachment may have a third surface configured to face towards the targeted site, and the third surface may have a surface area that is different from that of the first surface of the base plate.

In another example, the present invention is for a targeted pressure kit for providing targeted pressure to a targeted site. The targeted pressure kit includes a targeted pressure device having a base plate and at least two anchoring members fixedly coupled to the base plate. The base plate has a first surface configured to face towards the targeted site. The kit further includes a wrapping member. The targeted pressure device is configured for being attached to the targeted site by wrapping the wrapping member around or through the at least two anchoring members. Still further, the kit includes a pressure-enhancing attachment configured for attaching to the base plate and extending from the first surface of the base plate, and a storage plate to which the targeted pressure device and the pressure-enhancing attachment are removably coupled. The storage plate is configured for being coupled to a belt or a webbing system. The targeted pressure kit may further include a second pressure-enhancing attachment. The base plate, the pressure-enhancing attachment, and the second pressure-enhancing attachment may have different sized surface areas from each other. The targeted pressure kit may further include a hemostatic gauze pad attached to the first surface of the base plate and/or a wound side surface of the pressure-enhancing attachment.

In still another example, the present invention is for an auto-locking apparatus for controlling hemorrhage that enables a user to apply point pressure extremely rapidly with a single hand. In one embodiment, the inventive apparatus may be comprised of a mounting plate for mounting the apparatus over a wound site, an articulating strap attachment point that extends from the mounting plate, wherein the articulating strap attachment point may connect to a strap that secures the apparatus over the wound site, a coupling mechanism for coupling the mounting plate to a buckle on the strap that secures the apparatus over the wound site, wherein the coupling mechanism may permit the mounting plate and the buckle to freely rotate around each other when the mounting plate and the buckle are connected to the articulating strap attachment point, wherein the coupling mechanism ensures that the mounting plate remains parallel to the wound site when the apparatus is secured to a patient, a pressure applicator for applying point pressure to the wound site, wherein the pressure applicator extends through the mounting plate and being stabilized in a lateral direction by the mounting plate, an auto-locking mechanism enabling the pressure applicator to travel, upon application of downward force, towards the wound site when the apparatus is placed over the wound site, wherein the auto-locking mechanism prevents the pressure applicator from travelling away from the wound site when downward pressure is removed from the pressure applicator, and wherein the auto-locking mechanism comprising a locking tab, and a finger hold disposed on the mounting plate, wherein the finger hold enables a user to apply counter-force in a generally opposite direction to the downward force that may be applied to the pressure applicator, and wherein the counter-force enables a user to apply leverage when applying downward force to the pressure applicator.

A feature of the present invention is that it enables a user to apply point pressure rapidly. As described above, traditional point-pressure systems employ a screw-in system for slowly moving a pressure rod in a downward direction. This is a reliable method of pressure application. But, it takes time to apply pressure by turning a screw, especially in emergency situations where time is of the essence. The pressure application system of the present invention permits a user to freely slide a pressure applicator in a downward direction by simply applying pressure in a downward direction. The apparatus of the present invention is also comprised of an auto-locking mechanism, which enables the apparatus to maintain pressure even after the user stops applying downward pressure to the pressure applicator.

Another feature of the present invention is that it permits a user to apply pressure by using a single hand, and, as a result, a patient can self-apply the apparatus to his or her own wound in an effort to close it. In one embodiment, the inventive apparatus is comprised of a mounting plate that is further comprised of a finger hold. The finger hold permits a user to apply counter force to the pressure applicator. Without this feature, the user would end up applying pressure to the wound itself. Moreover, without this feature, single-handed application would not be possible.

Another feature of the present invention is that it can be used over any wound site on a patient's body. In one embodiment, the inventive apparatus is comprised of a freely rotating coupling mechanism for coupling the apparatus to straps. More specifically, the coupling mechanism permits the apparatus and/or the straps to rotate freely around each other. This enables a user to secure the straps around any portion of the patient's body, while ensuring that the mounting plate remains over the wound site and remains generally parallel to the wound site. As such, the pressure application maintains its position and applies appropriate downward pressure to occlude a blood vessel or a wound.

Another feature of the present invention is that it stabilizes the apparatus over a wound, which enables users to deploy the inventive apparatus in a field environment. The freely rotating coupling mechanism described above also ensures that the mounting plate remains parallel to the wound.

### BRIEF DESCRIPTION OF THE DRAWING FIGURES

The accompanying drawings illustrate several embodiments and, together with the description, serve to explain the principles of the invention according to the embodiments. It will be appreciated by one skilled in the art that the particular arrangements illustrated in the drawings are merely exemplary and are not to be considered as limiting of the scope of the invention or the claims herein in any way.

FIG. 1 illustrates, in accordance to one embodiment of the invention, an exemplary inventive apparatus.

FIG. 2A illustrates, in accordance to one embodiment of the invention, an exemplary apparatus that is connected to a patient's shoulder to apply point-pressure on the patient's subclavian artery.

FIG. 2B illustrates, in accordance with an embodiment of the invention, an exemplary apparatus that is attached to a patient's torso to apply point-pressure on the patient's subclavian artery.

FIG. 2C illustrates, in accordance with an embodiment of the invention, an exemplary apparatus that is attached to a patient's torso to apply point-pressure on the patient's abdominal aorta.

FIG. 3 illustrates an exemplary apparatus from another perspective, in accordance with an embodiment of the invention.

FIG. 4 illustrates an exemplary apparatus, in accordance with another embodiment of the invention.

FIG. 5 illustrates an exemplary mounting plate of the inventive apparatus in accordance with an embodiment of the invention.

FIG. 6A is a perspective view of an apparatus for applying point pressure, in accordance with one embodiment of the present invention.

FIG. 6B is an exploded view of the nesting pressure heads for the point pressure device in FIG. 6A, in accordance with one embodiment of the present invention.

FIGS. 7A and 7B are perspective and front views, respectively of a targeted pressure device for providing point or proximal pressure to a targeted site, in accordance with one embodiment of the present invention.

FIGS. 7C and 7D are plan views of the targeted pressure device in FIGS. 7A and 7B in use, in accordance with one embodiment of the present invention.

FIG. 8A is a top plan view of a targeted pressure device for providing point pressure to a targeted site, in accordance with one embodiment of the present invention.

FIG. 8B is a perspective view of the targeted pressure device from FIG. 8A and an associated pressure-enhancing attachment, in accordance with one embodiment of the present invention.

FIG. 8C is a side view of the targeted pressure device of FIGS. 8A and 8B with the pressure-enhancing attachment coupled thereto, in accordance with one embodiment of the present invention.

FIGS. 9A, 9B, and 9C are exploded, perspective, and top plan views, respectively, of a targeted pressure kit, in accordance with one embodiment of the present invention.

FIG. 10A is a side view of a targeted pressure device, in accordance with one embodiment of the present invention.

FIGS. 10B, 10C, and 10D are exploded, side, and top plan views, respectively, of a targeted pressure kit that includes the targeted pressure device of FIG. 10A, in accordance with one embodiment of the present invention.

### DETAILED DESCRIPTION

The inventor has conceived, and reduced to practice, an apparatus for applying point pressure. The apparatus may be used for a variety of use cases, including, for example, to occlude open blood vessels or open wounds, for therapeutic use, such as to apply pressure to acupressure points along a patient's body. Specifically, the inventive apparatus of the present invention is for a targeted pressure device for providing point or proximal pressure to a targeted site. The targeted pressure device includes a base plate having a first surface configured to face towards the targeted site and a second surface opposite of the first surface, wherein the second surface is configured to face away from the targeted site. The targeted pressure device further includes at least two anchoring members fixedly coupled to the second surface of the base plate and extending in a substantially perpendicular direction relative to the base plate. The anchoring members are configured for engaging with a wrapping member that is used to attach the targeted pressure device to the targeted site.

The invention is described by reference to various elements herein. It should be noted, however, that although the various elements of the inventive apparatus are described separately below, the elements need not necessarily be separate. The various embodiments may be interconnected and may be cut out of a singular block or mold. The variety of different ways of forming an inventive apparatus, in accordance with the disclosure herein, may be varied without departing from the scope of the invention.

Generally, one or more different embodiments may be described in the present application. Further, for one or more of the embodiments described herein, numerous alternative arrangements may be described; it should be appreciated that these are presented for illustrative purposes only and are not limiting of the embodiments contained herein or the claims presented herein in any way. One or more of the arrangements may be widely applicable to numerous embodiments, as may be readily apparent from the disclosure. In general, arrangements are described in sufficient detail to enable those skilled in the art to practice one or more of the embodiments, and it should be appreciated that other arrangements may be utilized and that structural changes may be made without departing from the scope of the embodiments. Particular features of one or more of the embodiments described herein may be described with reference to one or more particular embodiments or figures that form a part of the present disclosure, and in which are shown, by way of illustration, specific arrangements of one or more of the aspects. It should be appreciated, however, that such features are not limited to usage in the one or more particular embodiments or figures with reference to which they are described. The present disclosure is neither a literal description of all arrangements of one or more of the embodiments nor a listing of features of one or more of the embodiments that must be present in all arrangements.

Headings of sections provided in this patent application and the title of this patent application are for convenience only and are not to be taken as limiting the disclosure in any way.

Devices and parts that are connected to each other need not be in continuous connection with each other, unless expressly specified otherwise. In addition, devices and parts that are connected with each other may be connected directly or indirectly through one or more connection means or intermediaries.

A description of an aspect with several components in connection with each other does not imply that all such components are required. To the contrary, a variety of optional components may be described to illustrate a wide variety of possible embodiments and in order to more fully illustrate one or more embodiments. Similarly, although process steps, method steps, or the like may be described in a sequential order, such processes and methods may generally be configured to work in alternate orders, unless specifically stated to the contrary. In other words, any sequence or order of steps that may be described in this patent application does not, in and of itself, indicate a requirement that the steps be performed in that order. The steps of described processes may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to one or more of the embodiments, and does not imply that the illustrated process is preferred. Also, steps are generally described once per aspect, but this does not mean they must occur once, or that they may only occur once each time a process, or method is carried out or executed. Some steps may be omitted in some embodiments or some occurrences, or some steps may be executed more than once in a given aspect or occurrence.

When a single device or article is described herein, it will be readily apparent that more than one device or article may be used in place of a single device or article. Similarly, where more than one device or article is described herein, it will be readily apparent that a single device or article may be used in place of the more than one device or article.

The functionality or the features of a device may be alternatively embodied by one or more other devices that are not explicitly described as having such functionality or features. Thus, other embodiments need not include the device itself.

Techniques and mechanisms described or referenced herein will sometimes be described in singular form for clarity. However, it should be appreciated that particular embodiments may include multiple iterations of a technique or multiple instantiations of a mechanism unless noted otherwise. Alternate implementations are included within the scope of various embodiments in which, for example, functions may be executed out of order from that shown or discussed, including substantially concurrently or in reverse order, depending on the functionality involved, as would be understood by those having ordinary skill in the art.

### Overview

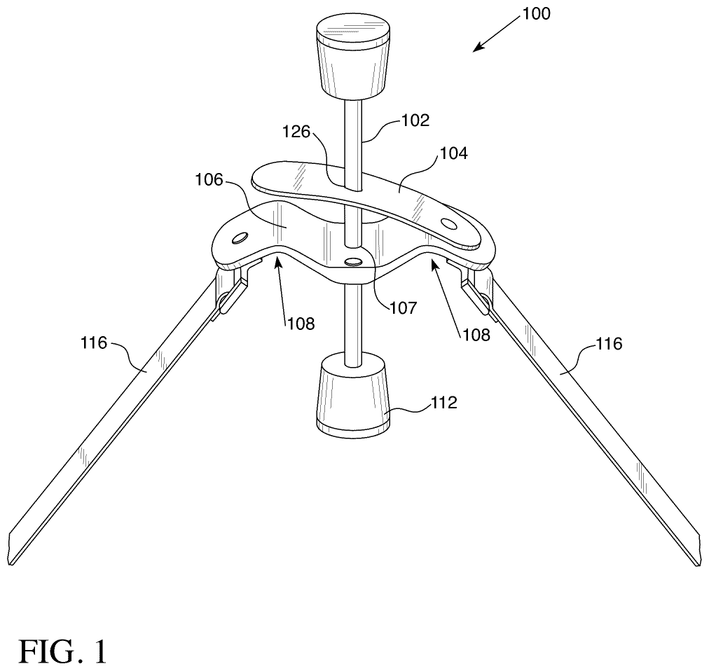

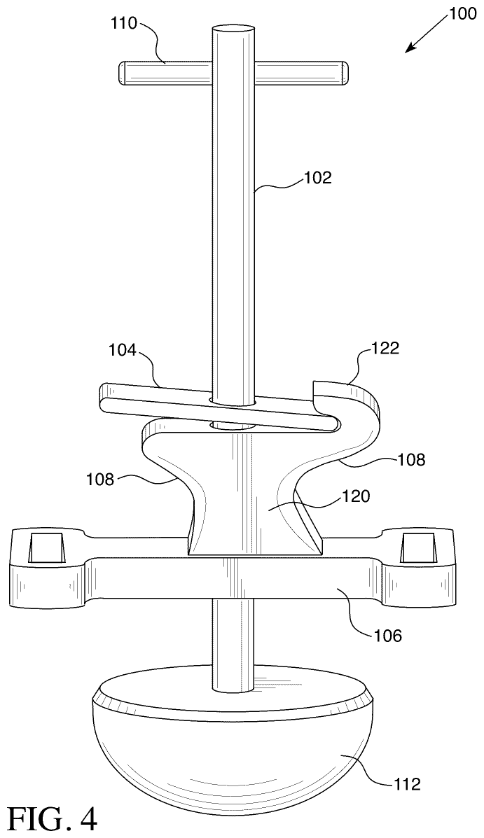

FIGS. 1 and 4 illustrate two exemplary embodiments of the inventive apparatus 100 disclosed herein. The inventive apparatus 100 is comprised of a mounting plate 106, articulating strap attachment point(s) 114, a pressure applicator 102, a locking tab 104, finger hold(s) 108, and a hemorrhage stopper 112. The apparatus 100 may be connected to straps 116 via the mounting plate 106 and the articulating strap attachment point(s) 114. FIG. 4 illustrates, in accordance with another exemplary embodiment of the invention, a tab extension portion 120, a tab holder 122, and grip support wings 110.

The various elements assemble together to form the inventive apparatus 100. The entire apparatus 100 may be placed over a wound. Once the apparatus 100 is properly placed, the pressure applicator 102 may be extended, by application of force, in a downward direction towards an open wound or blood vessel. In one embodiment of the invention, the pressure applicator 102 may extend downward until the hemorrhage stopper 112 contacts the open blood vessels and occludes it to prevent it from bleeding further. The finger hold(s) 108 assist a user in applying downward pressure by permitting the user to apply counter-force in an opposite direction (i.e. in an upward direction) to apply leverage to the pressure applicator 102.

The locking tab 104 engages the pressure applicator 102 when external downward pressure is removed from the pressure applicator 102 to prevent the pressure applicator 102 from extending upward or away from the open vessel. The locking tab 104 of the present invention permits the pressure applicator 102 to travel in a downward direction (i.e. toward a wound) upon application of downward force. But it prevents the pressure applicator 102 to travel in an upward direction unless a portion of the locking tab 104 is activated by application of downward force.

In this manner, the apparatus 100 ensures that pressure is maintained on the wound or the injured blood vessel, which prevents the patient from bleeding further. The articulating strap attachment point(s) 114 may be used to connect the apparatus to straps 116 that may be secured against the patient's body. In this manner, the apparatus 100 of the present invention may be used to prevent a patient from bleeding out due to an open wound or injured blood vessels.

The mounting plate 106 of the present invention is designed to be placed above a wound site and/or one or more open blood vessels. The mounting plate 106 serves as the foundation for connecting the various components of the inventive apparatus 100 described herein. Moreover, the mounting plate 106 also serves to stabilize the apparatus 100 over a wound side and serves to redistribute the forces that may be applied by one or more straps 116 when the straps 116 are attached to a patient and the mounting plate 106.

FIGS. 1 and 4 illustrate two exemplary mechanisms for connecting the mounting plate 106 to the locking tab 104. In one exemplary embodiment, as illustrated in FIG. 1, the mounting plate 106 is integrally attached to locking tab 104 via retention device. A variety of different retention devices may be used without departing from the scope of the invention, including, but not limited to a screw, a post, a bracket, etc. As illustrated in FIG. 1, the retention device may connect the mounting plate 106 and the locking tab 104 in a manner that the locking tab 104 may be moved in a downward direction by depressing the locking tab 104 at an end that is not connected to the mounting plate 106, thereby permitting the locking tab 104 to be placed in a plane that is substantially orthogonal to the pressure applicator 102. As described in greater detail below, once the locking tab 104 is in a substantially orthogonal plane to the pressure applicator 102, the aperture or opening that is formed within the locking tab 104, and through which the pressure applicator 102 is disposed, becomes substantially orthogonal to the pressure applicator 102 and permits the pressure applicator 102 to freely slide through the locking tab's aperture or opening and the mounting plate's aperture or opening 107 through with the pressure applicator 102 is disposed.

In another embodiment of the invention, the locking tab 104 may be attached to the mounting plate via an integrally formed tab extension portion 120 and tab holder 122. In other embodiments, the tab extension portion 120 and tab holder 122 may not be integrally formed, but may be attached via an attachment mechanism. As illustrated in FIG. 4, for example, the tab extension portion 120 may extend from the top surface of the mounting plate 106 (i.e. the surface that is faced away from the wound when the apparatus 100 is placed over the wound). A tab holder 122, which may be integrally formed or attached to the tab extension portion 120 may be comprised of a slot to accept the locking tab 104 and mate with it. As illustrated in FIG. 4, the tab holder 122 may mate with the locking tab 104 in a manner that the locking tab 104 may be moved in a downward direction by depressing the locking tab 104 at an end that is not connected to the tab holder 122, thereby permitting the locking tab 104 to be placed in a plane that is substantially orthogonal to the pressure applicator 102. As described in greater detail below, once the locking tab 104 is in a substantially orthogonal plane to the pressure applicator 102, the aperture or opening that is formed within the locking tab 104, and through which the pressure applicator 102 is disposed, becomes substantially orthogonal to the pressure applicator 102 and permits the pressure applicator 102 to freely slide through the locking tab's aperture or opening and the mounting plate's aperture or opening through with the pressure applicator 102 is disposed.

The mounting plate 106 may also be comprised of articulating strap attachment point(s) 114, which serve to couple the mounting plate 106 to straps 116 that may be attached around a patient. In one embodiment of the invention, the one or more articulating strap attachment point(s) 114 may extend downward from the bottom surface of the mounting plate 106 (i.e. the surface that faces the wound site when the apparatus 100 is placed over a patient's wound).

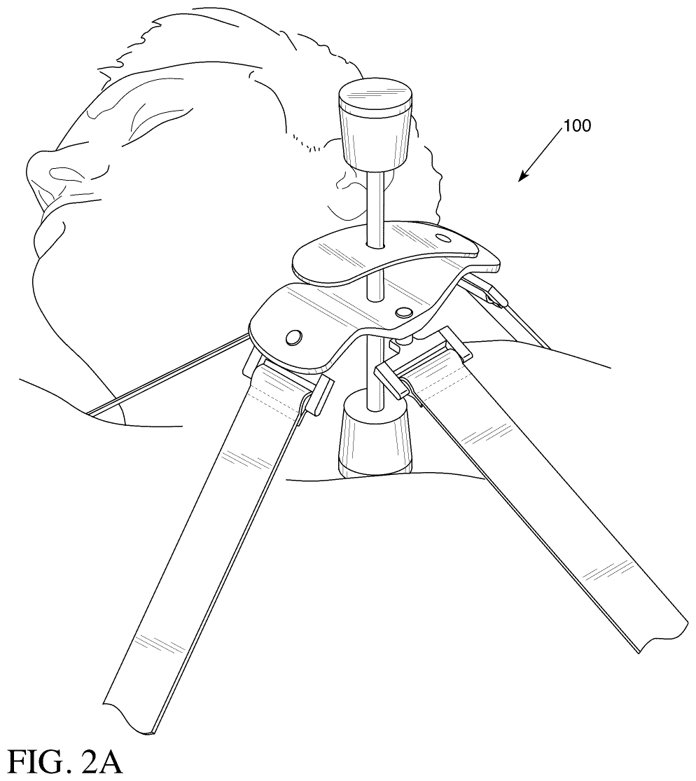

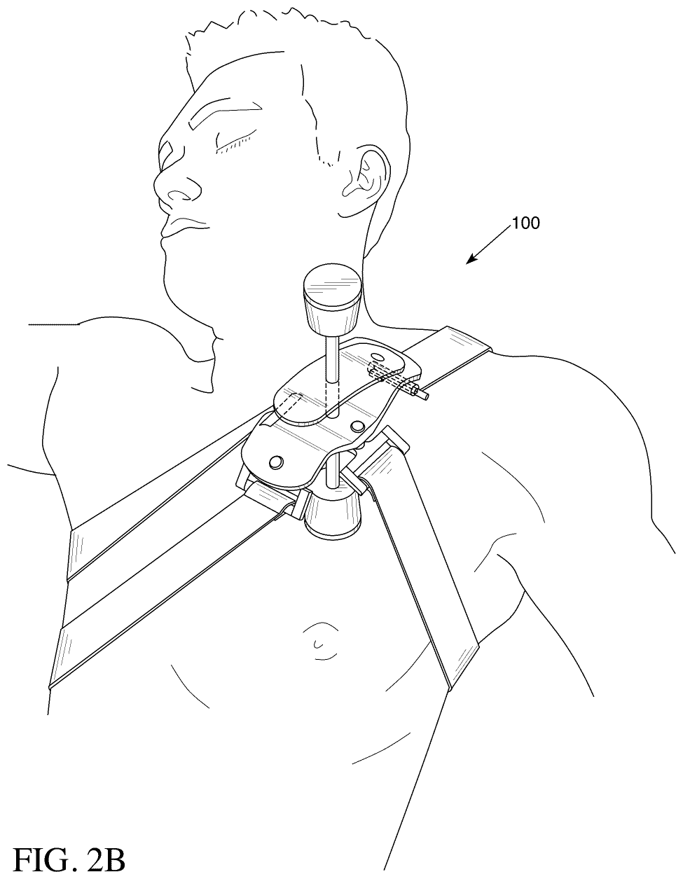

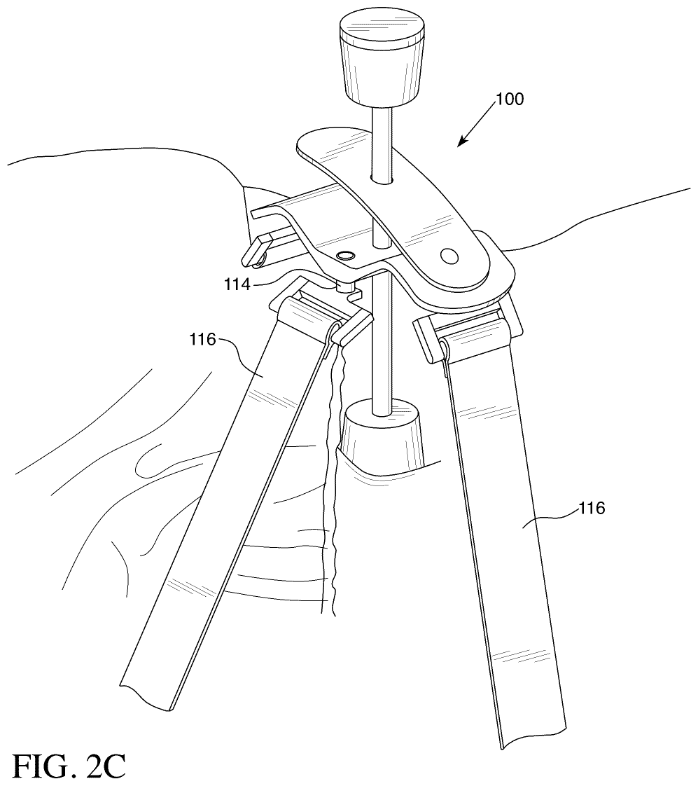

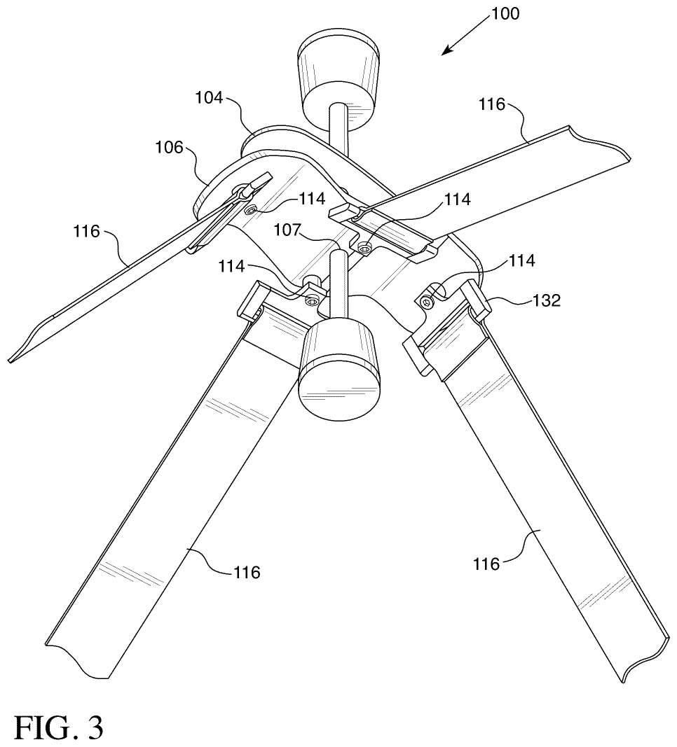

As illustrated in FIGS. 2C and 3, the articulating strap attachment point(s) 114 extend downward from the bottom surface of the mounting plate 106 (i.e. the surface that faces the wound site when the apparatus 100 is placed over a patient's wound). In accordance with an embodiment of the invention, the articulating attachment point(s) 114 form a coupling mechanism to permit a portion of one or more straps 116 to connect to the mounting plate 106. Specifically, as illustrated in FIG. 3, the articulating attachment point(s) 114 couple to buckles 132 that may be further connected to straps 116. In one embodiment of the invention, the strap buckles 132 are comprised of an aperture or an opening. The buckle's aperture may be placed over an articulating strap attachment point 114, and the attachment point 114 may thereafter be locked by a screw, a bolt mechanism, or another similar system to ensure that the straps do not become inadvertently disconnected from the mounting plate 106. In one embodiment, the diameter of the buckle's 132 aperture may be slightly larger than the exterior diameter of the articulating strap attachment point(s) 114, wherein the aperture is substantially circular and the articulating strap attachment point(s) 114 are substantially cylindrical. This feature of the present invention permits the strap buckles 132 and the mounting plate 106 to rotate around each other along a central axis.

The coupling mechanism of the present invention enables the apparatus to securely fit over a variety of different portions of a patient's body and/or body parts, and permits the apparatus 100 to remain substantially parallel to the wound. FIGS. 2A, 2B, and 2C highlight this point by illustrating the apparatus 100 as being securely fastened to a patient's throat and upper body, as shown in FIG. 2A, over the patient's chest to occlude the patient's subclavian artery, as illustrated in FIG. 2B, and over the patient's stomach to occlude the abdominal aorta, as illustrated in FIG. 2C. These figures are exemplary to illustrate how the apparatus may fit over a variety of different body parts and wound sites. However, the figures and the description herein is not limited to placement over these particular body parts or arteries. The apparatus 100 may be used over a variety of different parts of a user's body, including, but not limited to arteries in the pelvis where the pelvic bifurcation exists, a rotation at the attaching points to a parallel configuration with both around the waist is necessary to apply pressure to the abdominal vessels, the straps can then be rotated to be slightly off axis to allow one strap around the chest and one over the shoulder to apply pressure in the axilla. The same would be necessary to apply pressure to the subclavian artery. The rotation permits the apparatus 100 to apply pressure in nontraditional locations such as heavy venous bleeding from lower abdominal mid axillary wounds or buttocks wounds or caring for large patients.

In one embodiment, the apparatus 100 may be comprised of four articulating strap attachment points 114. The four attachment points 114 may be disposed along the perimeter of the mounting plate's bottom surface. The four attachment points 114 redistribute the compressive force that may be applied by the straps when they are securely fastened over a portion of the patient's body. In one embodiment, the four attachment points 114 enable the straps to form an “X” pattern to cross under the apparatus to stabilize the apparatus when attached to a patient.

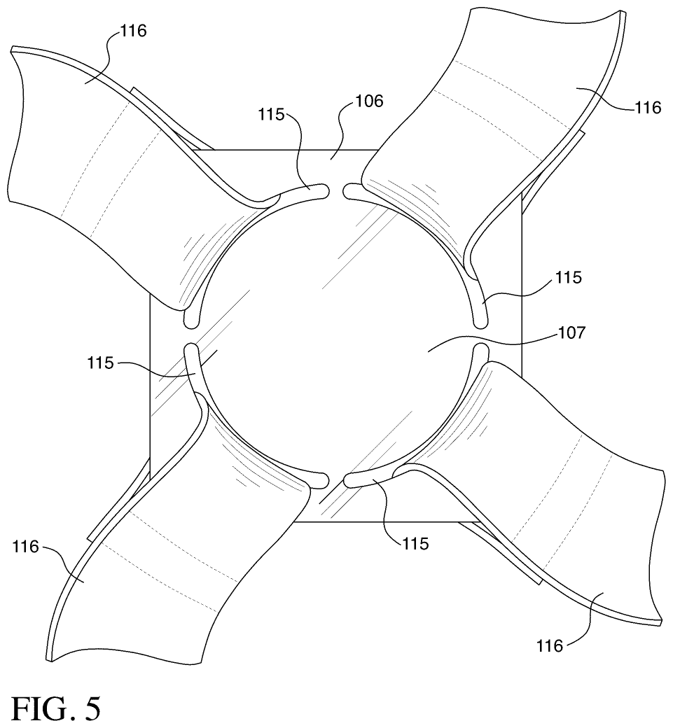

Now referring to FIG. 5, which illustrates another exemplary mounting plate with an attachment mechanism for attaching straps 116 to the apparatus 100. As illustrated, the mounting plate 106 may be comprised of one more slots 115. The straps 116 that secure the apparatus 100 around a patient's body can be fed through the slots 115 to connect the straps 116 to the mounting plate 106. The slots 115 may extend around the outer perimeter of the mounting plate 106 to permit the straps 116 to slide from one end of the slot 115 to another. This configuration permits a user to secure the apparatus 100 around a variety of different locations on a patient's body. For example, if one or more straps need to extend at a particular angle from the mounting plate 106 to be placed securely around the patient's body, then the straps may be sufficiently moved within the slot 115 to ensure that the apparatus 100 is properly secured against the patient's body.

This stability enhancing feature of the present invention (i.e. the features disclosed herein with regards to the mounting plate 106 and its connection to straps 116) also eliminates the need to carry a variety of different instruments to stop the bleeding at a variety of different portions of the patient's body. This is especially beneficial in emergency care settings where it is not economical (packing of bag space) or realistic to carry multiple devices in varying strap configurations to treat all the types of bleeding from different sites.

The stability enhancing features of the present invention also eliminate the need for a support plate, which is typically used with point-pressure systems. Traditional support plates were believed to be necessary and an integral part of point-pressure systems. However, the inventor has identified a new system that eliminates the need for such components, and more importantly, reduces the number of items that a user or medical professional has to deploy and attach to the patient, especially when time is of the essence.

The stability enhancing features of the present invention enables the apparatus 100 to be used with a traditional tourniquet without requiring additional stability plates, etc. In one embodiment of the invention, the apparatus 100 of the present invention may be attached to a tourniquet. In these instances, the buckle closure system of a traditional tourniquet may be replaced with the apparatus 100 for the application of point pressure.

In some embodiments of the invention, the mounting plate 106 is also comprised of at least one aperture or opening 107 through which the pressure applicator 102 may slide in an upward or a downward direction. The mounting plate aperture or opening 107 is discussed in greater detail below in reference to FIGS. 1 and 4.

The mounting plate 106 may also be comprised of one or more finger hold(s) 108. In one embodiment of the invention, the finger holds 108 may be integrally formed on the bottom surface of the mounting plate 106 (i.e. the surface facing a wound when the apparatus 100 is disposed above the wound or an open blood vessel). The finger holds 108 enable a user to apply counter-pressure (or upward force) to the apparatus 100. In one embodiment, the user may apply upward pressure on the finger hold(s) 108 and—at the same time—apply downward pressure on the pressure applicator 102 with the palm of his or her hand to slide the pressure applicator 102 in a downward direction. In one embodiment, applying downward force to pressure applicator 102 in a downward direction causes the locking tab 104 to disengage, which permits the pressure applicator 102 to travel in a downward direction (i.e. in the direction of the wound).

Referring now to the finger hold(s) 108 as illustrated in FIGS. 1 and 4 more particularly, which permit a user to apply counterforce when applying downward force on the pressure applicator 102. In one exemplary embodiment, the finger hold 108 is molded into the mounting plate 106. The finger hold 108 allows the user to apply counter force to the upper stop. The finger hold 108 can also contain an aperture through which the rod is fed through, as well as be an affixing point for the lock tab.

The opposing force applied to the upper stop and finger hold 108 moves the rod through the lock tab and support base. Without this finger hold 108, downward force applied to the upper stop would move the device as a unit due to strapping being non-rigid and thus not force the rod through the lock tab and support base. Indeed, without the finger hold(s) 108 the apparatus 100 may not be able to apply sufficient force to extend the pressure applicator 102 to adequately stop the bleeding, while, at the same time, not cause damage to nearby tissue/wound, which could happen if the mounting plate 106 is pushed down on the wound site without application of counter-force.

The finger hold(s) 108 of the present invention also permit the apparatus 100 to meet several military spec requirements or desired attributes. For example, the strategically placed finger hold(s) 108, as illustrated in FIG. 1 permits a patient to apply pressure on his or her wound. In other words, it is possible to use the device single handedly as enabled by the finger holds(s) 108. This feature is beneficial in any strapping configuration and additionally allows the user to apply the device to themselves in some instances, and allows for single hand tightening.

Any number of finger hold(s) 108 may be used without departing from the scope of the invention.

The locking tab 104 may connect to the mounting plate 106 or extension portions that are connected to the mounting plate, as illustrated in FIGS. 1 and 4, respectively. In one embodiment, the locking tab 104 is simply a plate with an aperture 126. The plate, in its normal or resting configuration rests at an angle that is offset from a plane that is orthogonal to the pressure applicator 102. The offset angle applies friction and pressure on the pressure applicator 102 and prevents the pressure applicator from moving in an upward or downward direction relative to the mounting plate 106. When a user wishes to extend the pressure applicator 102 in either direction, the user may depress the locking tab 104 at the end that is not connected, directly or indirectly, to the mounting plate 106. Pressing the locking tab 104 in such a manner, aligns the aperture 126 to the pressure applicator 102 (i.e. places the aperture 126 in a plane that is substantially orthogonal to the pressure applicator 102). In one embodiment of the invention, the locking tab 104 automatically depresses whenever the pressure applicator 102 is also depressed by application of force by a user.

The locking mechanism described herein permits rapid application of pressure at a wound site. Unlike traditional prior art systems, which apply pressure slowly or gradually, the auto-locking mechanism of the present invention permits users to apply pressure extremely rapidly and by simply applying downward pressure on the pressure applicator. Moreover, unlike other pressure application systems, which are unreliable in transport scenarios and the like, the pressure application system of the present invention permits universal use in a variety of different combat, transport, and other scenarios.

The present disclosure is also for a method of using the apparatus 100 disclosed herein. The method is comprised of placing the apparatus 100 over a wound or an open blood vessel, attaching the apparatus 100 to the straps that may be disposed around a patient's body, applying downward pressure on the pressure applicator, applying counter pressure (or upward pressure) to the mounting plate 106 via provided finger holds 108.

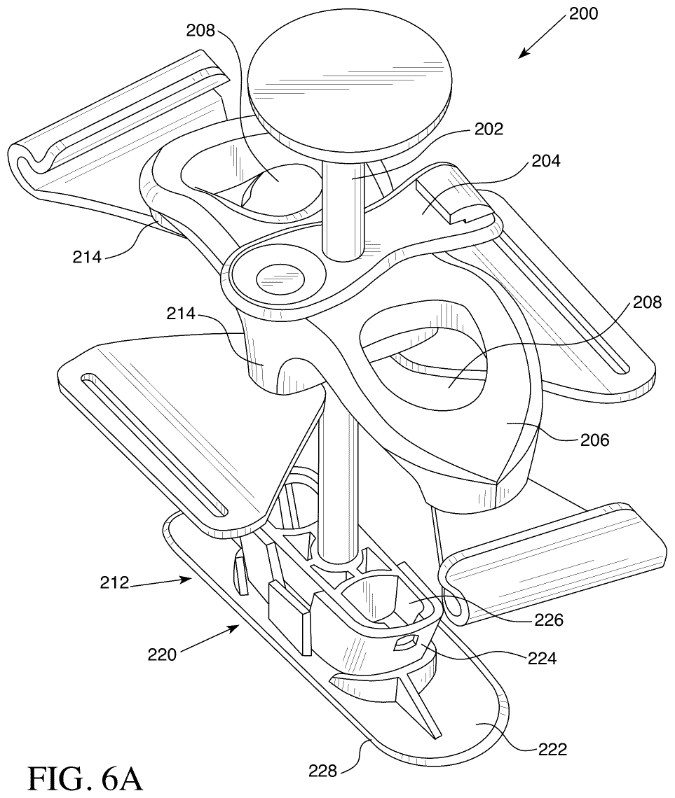

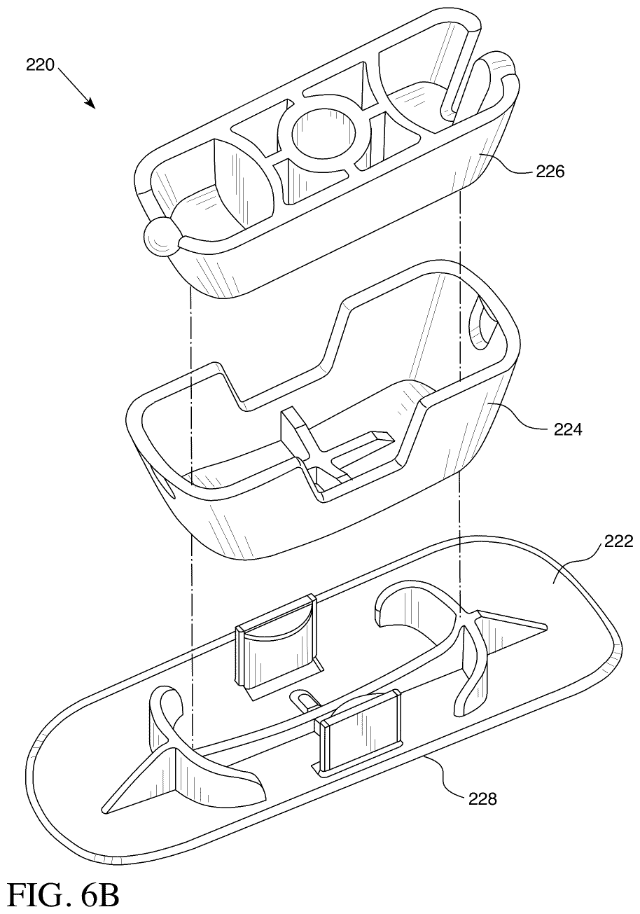

In the previous examples, the hemorrhage stopper 112 is relatively small for applying pressure to a small targeted area. In another example, an apparatus for applying targeted pressure to a wound site includes a larger plate for applying pressure to a larger area. The apparatus may further include a nested head system that allows a user to choose an appropriate size for the hemorrhage stopper at the distal end of the apparatus. As shown in FIGS. 6A and 6B, an apparatus 200 for applying targeted pressure to a wound site includes a mounting plate 206, articulating strap attachment points 214, a pressure applicator 202, a locking tab 204, finger holds 208, and a hemorrhage stopper 212. In this manner, the apparatus 200 is similar to the exemplary apparatus discussed previously herein. However, the hemorrhage stopper 212 has a larger surface area than the previous apparatuses disclosed herein. The apparatus 200 may further include a nested head system 220 that provides a plurality of options for sizes so that the user can choose an appropriate size depending on the surface area and/or location of the wound site.

The nested head system 220 allows a user to choose which size is needed for the hemorrhage stopper, depending on the size and/or location of the wound. The nested head system 220 may include an elongated curved plate 222 having the largest surface area, a medium sized head 224 that is configured to nest inside of the curved plate 222, and a small sized head 226 that is configured to nest inside of the medium sized head 224. The different sizes of the heads 222, 224, 226 of the hemorrhage stopper 212 may be coupled together in a nesting configuration with a snap fit mechanism, magnets, friction fit, or the like.

The apparatus 200 is not limited to the nested head system 220 shown in FIGS. 6A and 6B. The apparatus may alternatively include a nested head system having 2, 4, 5, or more different sizes of nested heads. In another example, the apparatus 200 may include only the elongated curved plate 222 and not have a nested head system at all.

The elongated curved plate 222 is advantageous for controlling stability and for increasing the margin of error during placement of the apparatus 200. The elongated curved plate 222 includes a convex surface 228 that is positioned in direct contact with the wound site when the apparatus 200 is in use.

Similar to the examples discussed above, the distance between the convex surface 228 of the curved plate 222 and the straps or the strap attachment points 214 is adjustable. When the pressure applicator 202 is pressed downward on the wound site and away from the mounting plate 206, the distance between the strap attachment points 214 and the hemorrhage stopper 212 is increased. Conversely, when the locking tab 204 is released and the pressure applicator 202 is allowed to move away from the wound site and proximally relative to the mounting plate 206, the distance between the strap attachment points 214 and the hemorrhage stopper 212 is decreased. Increasing the distance between the strap attachment points 214 and the hemorrhage stopper 212 causes increased pressure at the wound site. That is, the pressure applied by the curved plate 222 directly on the wound site is increased by pressing the pressure applicator 202 and increasing the distance between the strap attachment points 214 and the hemorrhage stopper 212.

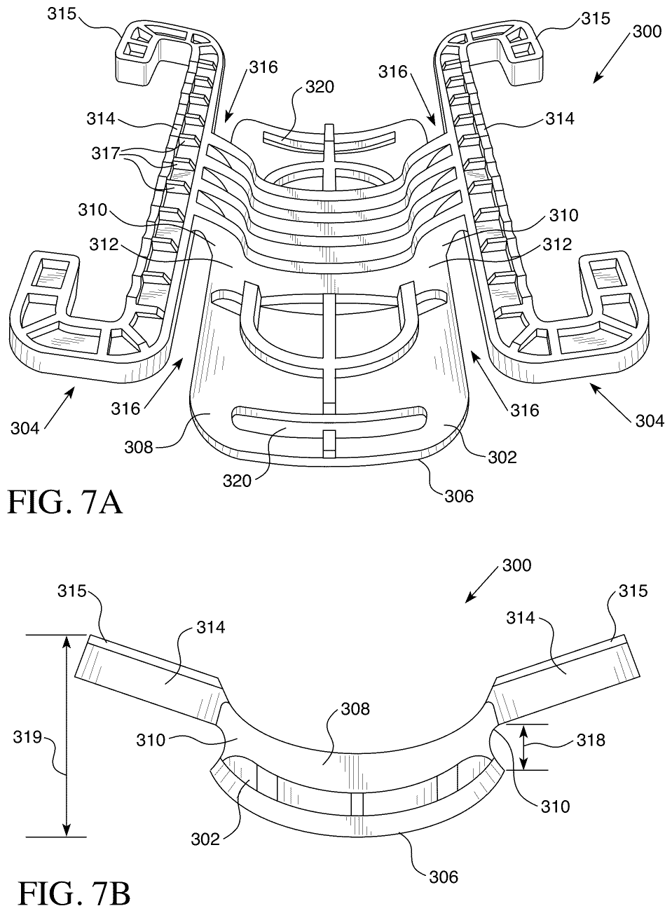

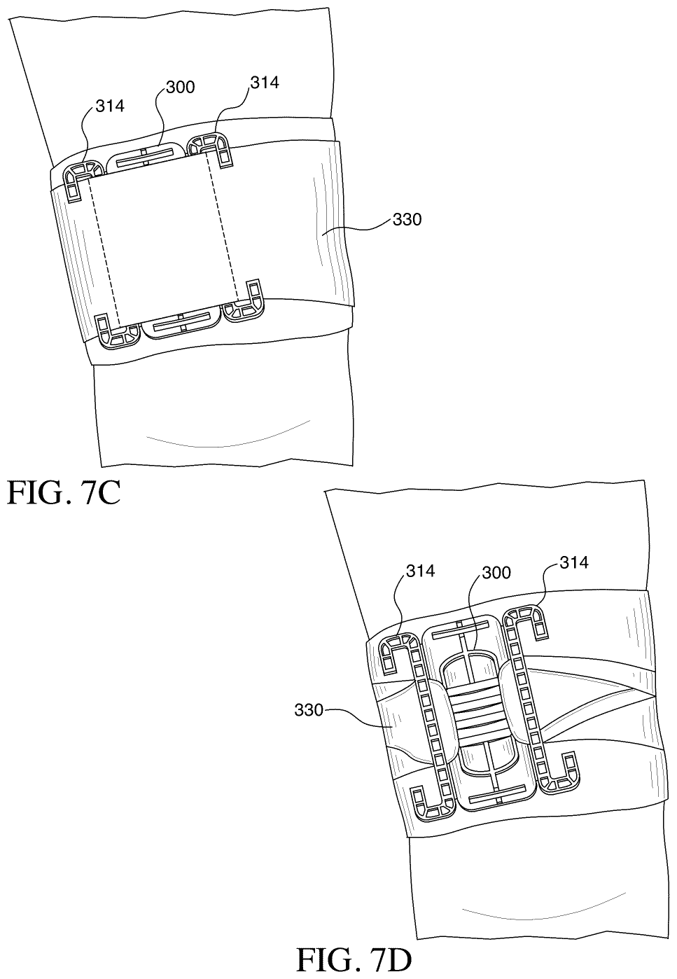

In yet another example, a hemorrhage control device for applying targeted pressure to a targeted site includes a base plate and anchoring members that are fixedly coupled to the base plate at a fixed distance. As such, this exemplary device 300, shown in FIGS. 7A-7B, has a fixed height, unlike the previous examples, which have an adjustable height between the pressure applicator and the strap attachment points. The targeted pressure device 300 for applying targeted pressure includes a curved base plate 302 and anchoring members 304 that are fixedly coupled to the base plate 302 with a fixed distance between them. The base plate 302 and the anchoring members 304 may be made of a single, unitary piece. The base plate 302 is configured for interfacing with the wound site and applying increased targeted pressure to the wound site. The device 300 is held in place at the wound site with bandages, wraps, tourniquets, or other such wrapping members that can be wrapped around or threaded through the anchoring members 304. The targeted pressure device 300 may be positioned at a targeted site for controlling bleeding. The targeted site may be a wound site or may be proximal to a wound site.

The base plate 302 has a first surface 306 configured to face towards the targeted site, and a second surface 308 configured to face away from the targeted site. The base plate 302 may be curved, as shown, so that the first surface 306 is convex and the second surface 308 is concave. This curved configuration of the base plate 302 provides enhanced stability and minimizes the likelihood of the device 300 being inadvertently shifted out of its desired position. However, the device 300 is not limited to having a curved base plate, and the base plate 302 may alternatively be arched, flat, domed, or the like to conform to different surface areas of a targeted site. The relatively large surface area of the base plate 302 allows for a margin of error when applying the device 300 to the targeted site. In other words, even if a user does not center the base plate 302 exactly on, or proximal to, the hemorrhaging blood vessel, the base plate 302 is large enough that targeted pressure will still be sufficiently applied to stop the bleeding.

In one example, the targeted pressure device 300 may include an attachment mechanism for attaching a doppler or ultrasound probe tip to the targeted pressure device 300. Doppler or ultrasound may be used to confirm that the targeted pressure device 300 is in a correct position for controlling hemorrhaging.

During use, the first surface 306 faces towards the targeted site while the anchoring members 304 are positioned at a fixed height above the targeted site. The first surface 306 may be in direct contact with the targeted site, or there may be intervening layers between the first surface 306 and the targeted site. For example, there may be gauze pads or bandages positioned between the targeted site and the first surface 306. In one example, the first surface 306 includes a hemostatic gauze pad fixedly coupled thereto so that the hemostatic gauze pad is in direct contact with the targeted site. In another example, the targeted pressure device 300 is affixed to a wrapping member, such as a bandage.

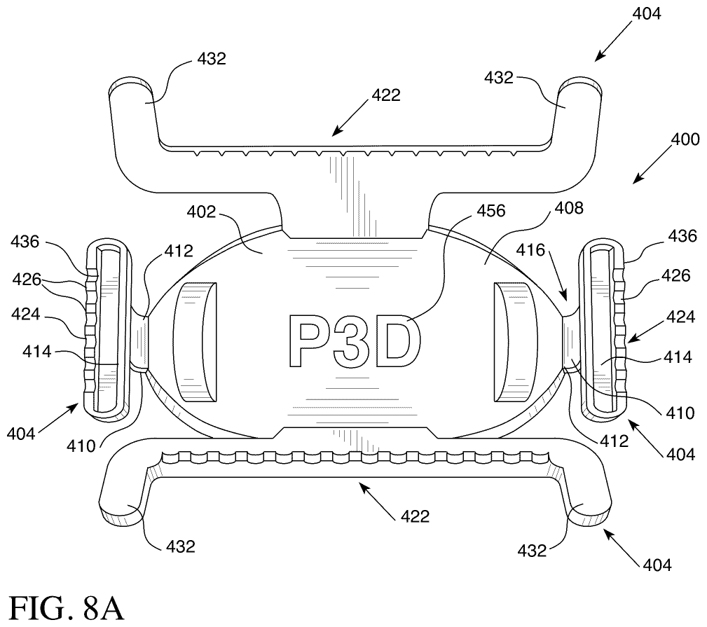

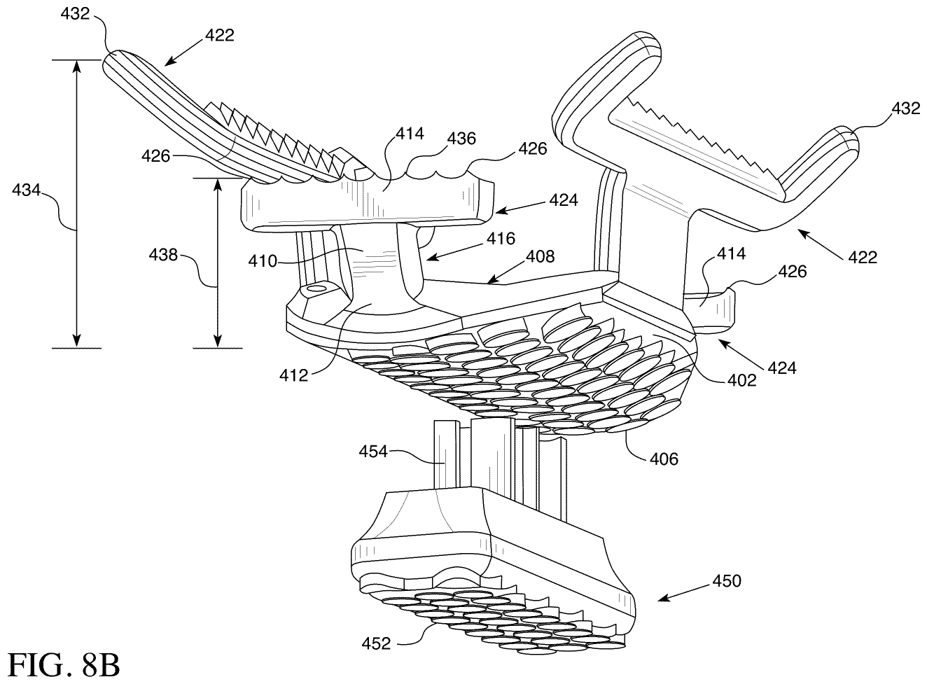

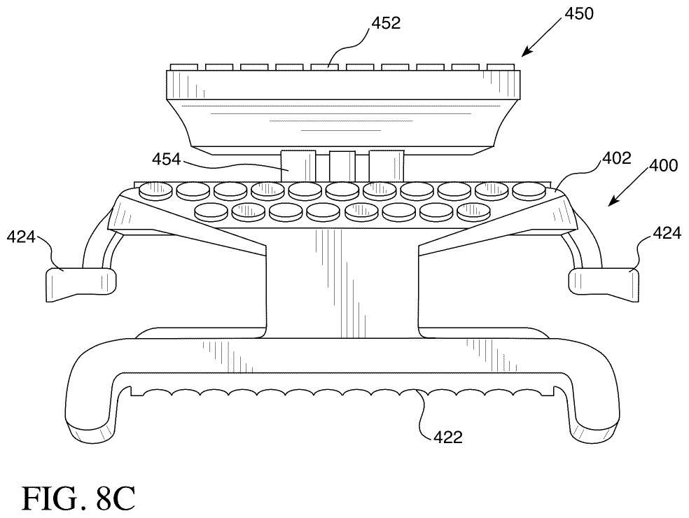

The anchoring members 304 include narrow distal portions 310 having distal ends 312 fixedly coupled to the base plate 302 and wide proximal portions 314 having proximal ends 315 that are a fixed distance 319 away from the base plate 302. The wide proximal portions 314 may include C-shaped brackets (as shown in FIG. 7A) or U-shaped brackets (as shown in FIGS. 8A and 8B). However, the device 300 is not limited to the proximal portion 314 having these shapes. Rather, the proximal portion 314 of the anchoring members 304 may have any desired shape that is configured to engage with a wrapping member, such as a bandage.

The distal portions 310 of the anchoring members 304 may be relatively narrow, thereby creating a channel 316 between the base plate 302 and the proximal portion 314. The distal portions 310 are configured as vertical columns that are substantially perpendicular to the base plate 302. The columns 310 create a space 316 between the base plate 302 and the brackets 314. Bandages can be wrapped around the columns 310 or routed through the brackets 314. As such, the anchoring members 304 engage with wrapping members, such as wraps, bandages, tourniquets, or the like, in order to apply increased pressure to the wound site through the base plate 302. The brackets 314 may include protruding teeth 317 or roughening ribs in order to increase the friction between the anchoring members 304 and the wrapping member. Further, the base plate 302 may include a non-smooth texture to increase the friction between the base plate 302 and the wound site to thereby prevent the plate 302 from slipping out of place. For example, the first surface 306 of the base plate 302 may include a rough, bumpy, or other such friction-enhancing texture.

The distal portions 310 of the anchoring members 304 may be smooth and radiused to allow a wrapping member to slide freely relative to the distal portion 310. The height 318 (shown in FIG. 7B) of the distal portion 310 is fixed and can be any desired height, depending on the amount of pressure that is desired. Similarly, the distance 319 between the first surface 306 of the base plate 302 and the proximal end 315 of the anchoring member 304 is fixed and can be any desired distance, depending on the amount of pressure that is desired.

The apparatus 300 can be used with commercially available wraps and tourniquets. The apparatus 300 provides downward targeted pressure via the pressure plate 302. The apparatus 300 is made of a rigid material, such as polymer, plastic, metal, acrylic, resin, or the like.

The proximal portions 314 of the anchoring members 304 may be C-shaped or U-shaped channels that allow for material routing, central positioning, and slippage prevention, as well as increased downward targeted point pressure when wider material is routed on top of the channels as opposed to being routed through the channels. The channels have raised ridges to prevent material slippage on the upper section and in the channel itself.

The base plate 302 may additionally include elongated openings 320 on either end of the base plate 302. The apparatus 300 may be secured to a wound site using a strap or bandage threaded through the elongated openings 320.

As shown in FIGS. 7C and 7D, the targeted pressure device 300 can be attached to a wound site using conventional bandaging material. FIG. 7C depicts the apparatus 300 attached to a wound site by wrapping a bandage 330 around the targeted area with the apparatus 300 positioned between the bandage 330 and the wound site. The bandage 330 is secured in place relative to the apparatus by threading the bandage 330 through the C-shaped brackets 314 of the anchoring members 304. FIG. 7D depicts the apparatus 300 attached to a wound site by wrapping the bandage 330 around the reduced width of the columns 310 so that the bandage is secured in place between the base plate 302 and the brackets 314. In this manner, the columns 310 provide a channel for changing the wrapping direction of the bandage. For example, a bandage that is being wrapped in an anti-clockwise direction can be wrapped around the column 310 to change direction to wrap in a clockwise direction.

The targeted pressure device 300 provides more pressure to the wound site than the bandage alone. The bandage is attached to the anchoring members 304, thereby pressing the convex surface 306 of the base plate 302 into the wound site with increased pressure due to the fixed distance 319 between the wound side surface 306 and the anchoring members 304.

FIGS. 8A-8C depict another example of a targeted pressure device 400 that includes a curved base plate 402 fixedly attached to anchoring members 404. The base plate 402 and the anchoring members 404 may be made of a single, unitary piece. The device 400 can be applied to a wound site by wrapping a wrapping member around, through, or on top of the anchoring members 404.

The device 400 includes four anchoring members 404 and each anchoring member 404 is fixedly attached to a different side of the base plate 402. First and second anchoring members 422 are attached to opposite lateral sides of the base plate 402, and third and fourth anchoring members 424 are attached to opposite longitudinal ends of the base plate 402. The two different sets of anchoring members 422, 424 provide two different options for applying different amounts of pressure to a targeted site. The proximal ends 432 of the first and second anchoring members 422 are a first fixed distance 434 away from the base plate 402, while the proximal ends 436 of the third and fourth anchoring members 424 are a second fixed distance 438 away from the base plate 402. In the example depicted in FIGS. 8A and 8B, the first fixed distance 434 is greater than the second fixed distance 438. However, it will be readily apparent to one of ordinary skill in the art that the second fixed distance 438 may be greater than the first fixed distance 434 or that the first fixed distance 434 may be equal to the second fixed distance 438. One advantage of the first fixed distance 434 being different from the second fixed distance 438 is that the user will have options for applying different amounts of pressure.

First and second anchoring members 422 are substantially similar to the anchoring members 304 in FIGS. 7A-7D. Third and fourth anchoring members 424 include narrow distal portions 410 having distal ends 412 fixedly coupled to the base plate 402 and wide proximal portions 414 having proximal ends 436 that are a fixed distance away from the base plate 402. The wide proximal portions 414 are configured as horizontal bars having gripping teeth 426 or a similar rough texture for engaging with a wrapping member and holding the wrapping member in place. However, the device 400 is not limited to the proximal portion 414 having the horizontal bar shape. Rather, the proximal portion 414 of the anchoring members 424 may have any desired shape that is configured to engage with a wrapping member, such as a bandage.

The distal portions 410 of the anchoring members 424 may be relatively narrow, thereby creating a channel 416 between the base plate 402 and the proximal portion 414. The distal portions 410 are configured as vertical columns that are substantially perpendicular to the base plate 402. The columns 410 create a space 416 between the base plate 402 and the horizontal bars 414. Bandages can be wrapped around the columns 410 or applied to the top surface of the horizontal bars 414.

The anchoring members 404 engage with wrapping members, such as wraps, bandages, tourniquets, or the like, in order to apply increased pressure to the wound site through the base plate 402. The base plate 402 may include a non-smooth texture to increase the friction between the base plate 402 and the wound site to thereby prevent the plate 402 from slipping out of place. For example, the first surface 406 of the base plate 402 may include a rough, bumpy, or other such friction-enhancing texture.

The distal portions 410 of the anchoring members 424 may be smooth and radiused to allow a wrapping member to slide freely relative to the distal portion 410. The height of the distal portion 410 is fixed and can be any desired height, depending on the amount of pressure that is desired. Similarly, the distance 438 between the first surface 406 of the base plate 402 and the proximal end 436 of the anchoring member 424 is fixed and can be any desired distance, depending on the amount of pressure that is desired.

The base plate 402 has a first surface 406 configured to face towards the targeted site, and a second surface 408 configured to face away from the targeted site. The base plate 402 may be curved, as shown, so that the first surface 406 is convex and the second surface 408 is concave. This curved configuration of the base plate 402 provides enhanced stability and minimizes the likelihood of the device 400 being inadvertently shifted out of its desired position. However, the device 400 is not limited to having a curved base plate, and the base plate 402 may alternatively be arched, flat, domed, or the like to conform to different surface areas of a targeted site. The relatively large surface area of the base plate 402 allows for a margin of error when applying the device 400 to the targeted site. In other words, even if a user does not center the base plate 402 exactly on, or proximal to, the hemorrhaging blood vessel, the base plate 402 is large enough that targeted pressure will still be sufficiently applied to stop the bleeding.

In one example, the targeted pressure device 400 may include an attachment mechanism for attaching a doppler or ultrasound probe tip to the targeted pressure device 400. Doppler or ultrasound may be used to confirm that the targeted pressure device 400 is in a correct position for controlling hemorrhaging.

During use, the first surface 406 faces towards the targeted site while the anchoring members 422, 424 are positioned at fixed heights 434, 438 above the targeted site. The first surface 406 may be in direct contact with the targeted site, or there may be intervening layers between the first surface 406 and the targeted site. For example, there may be gauze pads or bandages positioned between the targeted site and the first surface 406. In one example, the first surface 406 includes a hemostatic gauze pad fixedly coupled thereto so that the hemostatic gauze pad is in direct contact with the targeted site. In another example, the targeted pressure device 400 is affixed to a wrapping member, such as a bandage.

The apparatus 400 can be used with commercially available wraps and tourniquets. The apparatus 400 provides downward targeted pressure via the pressure plate 402. The apparatus 400 is made of a rigid material, such as polymer, plastic, metal, acrylic, resin, or the like.

As shown in FIGS. 8B and 8C, the targeted pressure device 400 includes a pressure-enhancing attachment 450 that can be attached to the base plate 402 to provide an increased distance between the wound-side surface of the device 400 and the anchoring members 404. The pressure-enhancing attachment 450 includes a wound-side surface 452 and an attachment mechanism for engaging with the base plate 402. The wound-side surface 452 of the attachment 450 has a surface area that is different from the surface area of the base plate 402. In the example shown in FIG. 8B, the surface area of the attachment 450 is smaller than the surface area of the base plate 402. However, the surface area of the attachment 450 may alternatively be larger than or equal to the surface area of the base plate 402.

The attachment 450 may be removably attached to the base plate 402 using any known attachment mechanism. For example, the attachment 450 may be coupled to the base plate 402 by friction fit, snap fit, magnets, suction, tongue and groove, clamps, clips, adhesive, hook and loop fasteners, or the like. In the example shown in FIGS. 8B and 8C, the attachment 450 is attached to the base plate 402 with friction fit. That is, the attachment 450 includes a protrusion 454 and the base plate 402 includes an opening 456 (shown in FIG. 8A). The size and shape of the opening 456 mimics the size and shape of the protrusion 454 so that the protrusion 454 fits within the opening 456 in order to removably couple the attachment 450 to the base plate 402.

In one example, the attachment mechanism for attaching the pressure-enhancing attachment 450 to the base plate 402 includes a spring. For instance, there may be a spring pocket between the attachment 450 and the base plate 402. The spring would allow for some compression and adjustability.

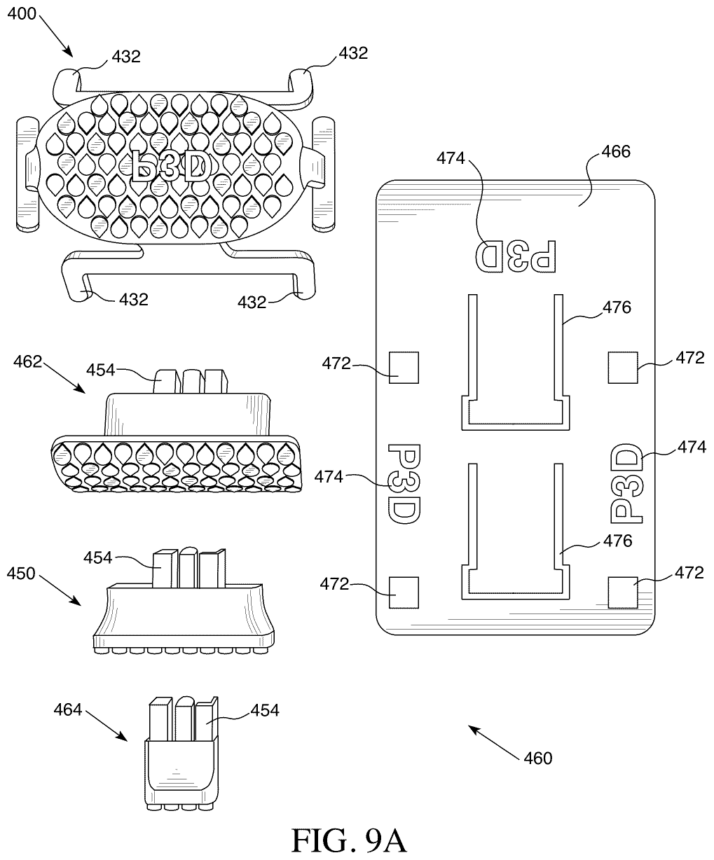

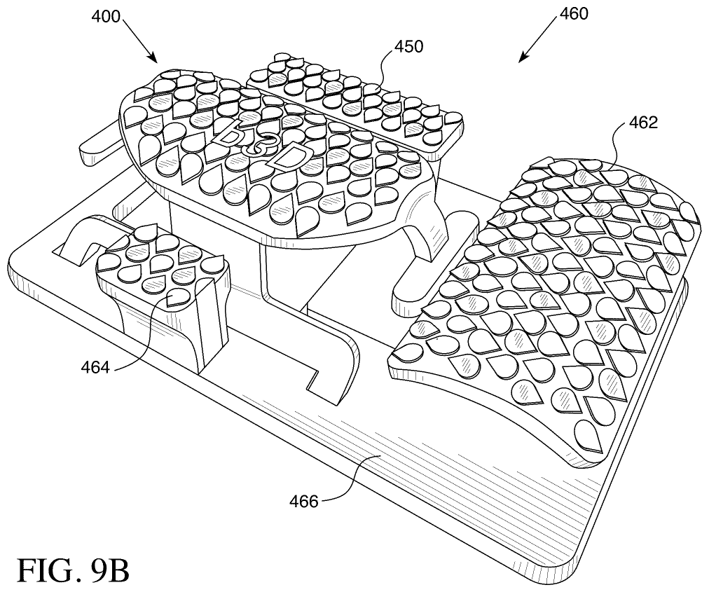

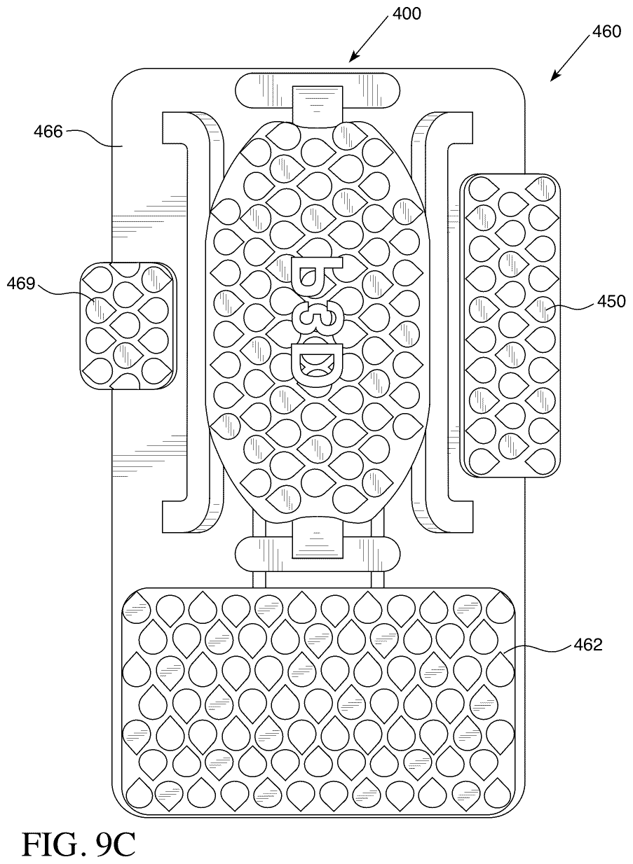

As shown in FIGS. 9A-9C, the targeted pressure device 400 and the attachment 450 may be provided together in a kit 460. The kit 460 may further include other pressure-enhancing attachments 462, 464 and a storage plate 466. The pressure-enhancing attachments 450, 462, 464 may each have a different size so that a user can choose which size of surface area will work best for the size of the wound. In other words, a larger surface area would be more desirable for a larger wound and a smaller surface area would be more desirable for a smaller wound.

The storage plate 466 is configured for holding all of the components of the kit 460 together for ease of use and storage. The targeted pressure device 400, pressure-enhancing attachments 450, 462, 464, and a wrapping member (not shown) may be attached to the storage plate 466.

As shown in FIG. 9A, the storage plate 466 may include openings 472 for engaging with the proximal ends 432 of the anchoring members 422 so that the targeted pressure device 400 is coupled to the storage plate 466 with a friction fit engagement. Similarly, the storage plate 466 includes apertures 474 that mimic the size and shape of the protrusions 454 on the pressure-enhancing attachments 450, 462, 464. Thus, the pressure-enhancing attachments 450, 462, 464 can be coupled to the storage plate 466 with a friction fit engagement. The storage plate 466 further includes central openings 476 surrounding T-shaped tabs in the central portion of the plate 466. The central openings 476 are configured for attaching the storage plate 466 to a belt or a webbing system, such as MOLLE.

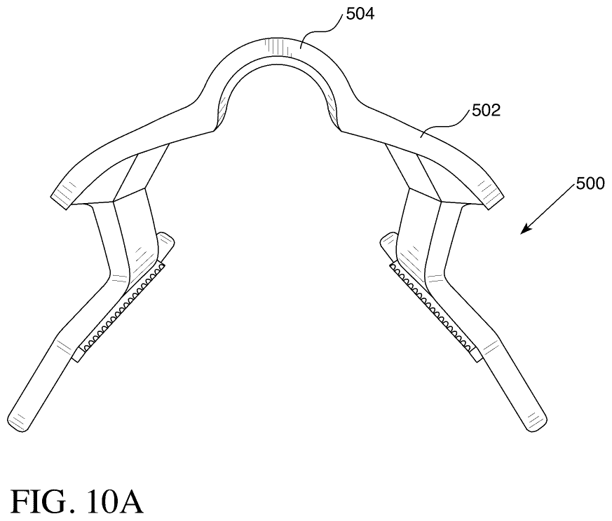

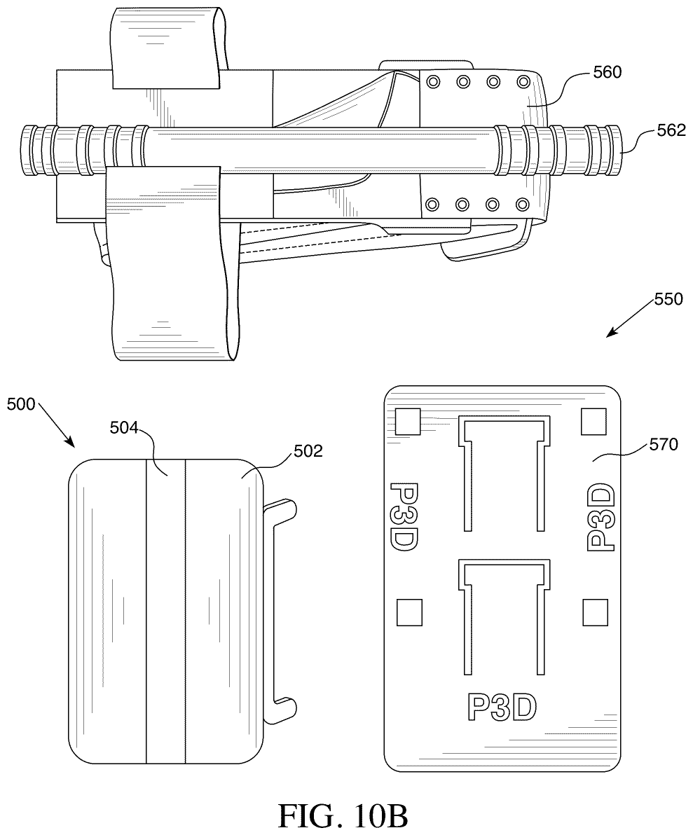

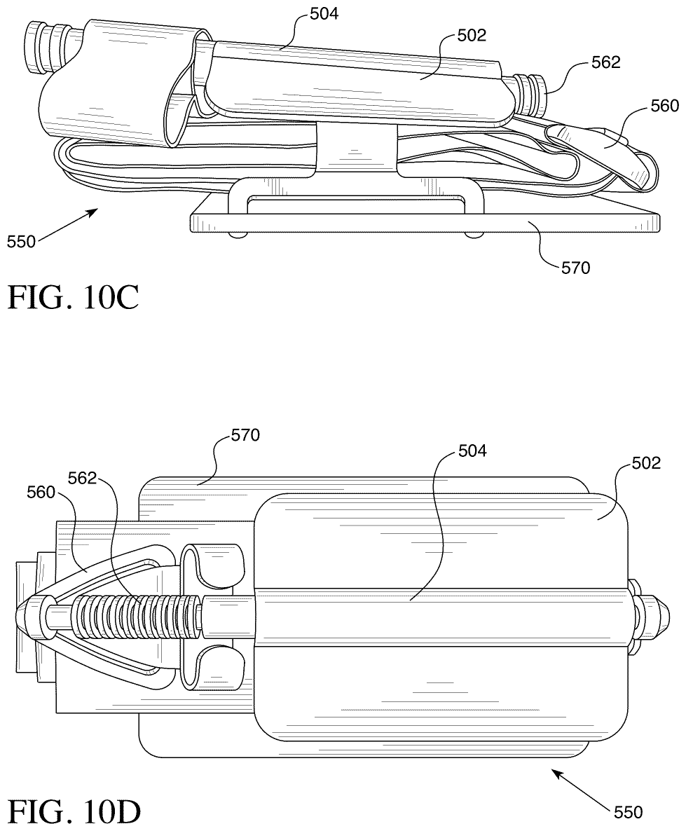

In another example, shown in FIGS. 10A-10D, the base plate 502 of the targeted pressure device 500 may have an arched portion 504 that extends the entire length of the plate 502. The arched portion 504 is sized and shaped to accommodate a tourniquet windlass being positioned therein. As shown in FIGS. 10B-10D, the targeted pressure device 500 may be provided in a kit 550 that includes a wrapping member in the form of a tourniquet 560 and a storage plate 570. When the kit 550 is assembled, as shown in FIGS. 10C and 10D, the tourniquet 560 fits in the space between the targeted pressure device 500 and the storage plate 570. The windlass 562 of the tourniquet 560 fits within the grooved portion 504 of the base plate 502.

### Additional Considerations

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and Bis false (or not present), A is false (or not present) and Bis true (or present), and both A and B are true (or present).

In addition, use of the “a” or “an” are employed to describe elements and components of the embodiments herein. This is done merely for convenience and to give a general sense of the invention. This description should be read to include one or at least one and the singular also includes the plural unless it is obvious that it is meant otherwise.

Upon reading this disclosure, those of skill in the art will appreciate still additional alternative structural and functional designs for a system and a process for creating an interactive message through the disclosed principles herein. Thus, while particular embodiments and applications have been illustrated and described, it is to be understood that the disclosed embodiments are not limited to the precise construction and components disclosed herein. Various apparent modifications, changes and variations may be made in the arrangement, operation and details of the method and apparatus disclosed herein without departing from the spirit and scope defined in the appended claims.

What is claimed is:

1. A targeted pressure device for providing point or proximal pressure to a targeted site, wherein the targeted pressure device comprises:
a base plate having a first surface configured to face towards the targeted site and a second surface opposite of the first surface, wherein the second surface is configured to face away from the targeted site; and
at least two anchoring members fixedly coupled to the second surface of the base plate and extending in a substantially perpendicular direction relative to the base plate, wherein each one of the anchoring members comprises:
a narrow distal portion having a distal end fixedly coupled to the second surface of the base plate; and
a wide proximal portion having a proximal end that is a fixed distance away from the base plate,
wherein the at least two anchoring members are configured for engaging with a wrapping member that is used to attach the targeted pressure device to the targeted site.

2. The targeted pressure device of claim 1, wherein the wide proximal portion comprises a bracket that is C-shaped or U-shaped, and wherein the bracket is configured for holding the wrapping member in place.

3. The targeted pressure device of claim 1, wherein the wide proximal portion comprises a horizontal bar that is configured for holding the wrapping member in place.

4. The targeted pressure device of claim 1, wherein the wide proximal portion comprises protruding teeth for gripping the wrapping member.

5. The targeted pressure device of claim 1, wherein the narrow distal portion comprises a smooth surface that allows the wrapping member to move relative thereto.

6. The targeted pressure device of claim 1, comprising four anchoring members, wherein a first anchoring member, a second anchoring member, a third anchoring member, and a fourth anchoring member are coupled to a first side of the base plate, a second side of the base plate, a third side of the base plate, and a fourth side of the base plate, respectively.

7. The targeted pressure device of claim 6, wherein the first anchoring member and the second anchoring member are positioned opposite of each other, and wherein the third anchoring member and the fourth anchoring member are positioned opposite of each other.

8. The targeted pressure device of claim 7, wherein the proximal ends of the first anchoring member and the second anchoring member are a first fixed distance away from the base plate, wherein the proximal ends of the third anchoring member and the fourth anchoring member are a second fixed distance away from the base plate, and wherein the second distance is different from the first distance.

9. The targeted pressure device of claim 1, wherein the first surface of the base plate has a rough texture for providing grip to the targeted site.

10. The targeted pressure device of claim 1, wherein the base plate is curved such that the first surface is convex and the second surface is concave.

11. The targeted pressure device of claim 1, further comprising a pressure-enhancing attachment configured to attach to the first surface of the base plate, such that a distance between the pressure-enhancing attachment and the proximal ends of the anchoring members is greater than the fixed distance between the base plate and the proximal ends of the anchoring members.

12. The targeted pressure device of claim 11, wherein the pressure-enhancing attachment and the base plate are configured to attach together with a friction fit attachment, snap fit attachment, adhesive, suction, tongue and groove, hook and loop fasteners, magnets, clamps, or clips.

13. The targeted pressure device of claim 12, wherein the base plate comprises an aperture having an aperture size and aperture shape, and wherein the pressure-enhancing attachment comprises a protrusion having a protrusion size and a protrusion shape that are substantially the same as the aperture size and the aperture shape, respectively, so that the pressure-enhancing attachment is coupled to the base plate with a friction fit between the aperture and the protrusion.

14. The targeted pressure device of claim 11, wherein the pressure-enhancing attachment comprises a third surface configured to face towards the targeted site, wherein the third surface has a surface area that is different from that of the first surface of the base plate.

15. The targeted pressure device of claim 1, wherein the base plate and the at least two anchoring members are formed as a single, unitary piece.

16. A targeted pressure kit for providing targeted pressure to a targeted site, wherein the targeted pressure kit comprises:
a targeted pressure device comprising a base plate and at least two anchoring members fixedly coupled to the base plate, wherein the base plate comprises a first surface configured to face towards the targeted site;
a wrapping member, wherein the targeted pressure device is configured for being attached to the targeted site by wrapping the wrapping member around or through the at least two anchoring members;
a pressure-enhancing attachment configured for attaching to the base plate and extending from the first surface of the base plate; and
a storage plate to which the targeted pressure device and the pressure-enhancing attachment are removably coupled, wherein the storage plate is configured for being coupled to a belt or a webbing system.

17. The targeted pressure kit of claim 16, further comprising a second pressure-enhancing attachment, wherein the base plate, the pressure-enhancing attachment, and the second pressure-enhancing attachment have different sized surface areas from each other.

18. The targeted pressure kit of claim 16, further comprising a hemostatic gauze pad attached to at least one of the first surface of the base plate and a wound side surface of the pressure-enhancing attachment.
